(19) United States
(12) Patent Application Publication
(10) Pub. No.: US 2015/0168362 A1
(43) Pub. Date: Jun. 18, 2015

(54) MICROFLUIDIC CHANNEL DETECTION SYSTEM

(71) Applicant: National Applied Research Laboratories, Hsinchu City (TW)

(72) Inventors: Che-Hsin LIN, Hsinchu City (TW); Jun-Jie WANG, Hsinchu City (TW); Ying-Zong JUANG, Hsinchu City (TW); Hann-Huei TSAI, Hsinchu City (TW); Hsin-Hao LIAO, Hsinchu City (TW)

(73) Assignee: National Applied Research Laboratories, Hsinchu City (TW)

(21) Appl. No.: 14/185,245

(22) Filed: Feb. 20, 2014

(30) Foreign Application Priority Data
Dec. 13, 2013 (TW) 102146116

(51) Int. Cl. G01N 33/00; G01N 27/00; H05K 3/00; B29C 65/00; B29C 43/02

(52) U.S. Cl. CPC G01N 33/0009; B29C 66/028; B29C 43/021; H05K 3/007; G01N 27/00; B29K 2083/005

(57) ABSTRACT

A microfluidic channel detection system for environmental or biomedical detection includes a chip having a first surface where a sensing region is located, a substrate having a recess for containing the chip, in which the first surface is exposed, a first inactive layer filling gaps between the chip and the substrate in the recess, so as to form a plane with the first surface of the chip, an electrical connection member electrically connected to the chip, a cover having a microfluidic channel and disposed on the plane. The flow path in the microfluidic channel is smooth, and further the measurement accuracy is improved via the plane formed by the first inactive layer and the first surface.

### CROSS REFERENCE TO RELATED APPLICATION

This application claims priority to Taiwan Application Serial Number 102146116, filed on Dec. 13, 2013, which are herein incorporated by reference.

### FIELD OF THE INVENTION

The present invention relates to a microfluidic channel detection system, in particular, a microfluidic channel detection system for environmental or biomedical detection by using a plane formed by a first inactive layer and a first surface of a chip so as to smooth the flow path within the microfluidic channel to increase the accuracy of measurements.

### BACKGROUND OF THE INVENTION

As shown in FIG. 1, this shows the structure of a conventional microfluidic channel detection system. In this conventional microfluidic channel detection system, a chip 2 for sensing is disposed on a substrate 3, and is connected to elements on the substrate 3 via wires. Because a sensing region on the chip 2 is slightly higher than the substrate 3, a whole cover 5 having the microfluidic channel 6 is disposed on top of the chip 2 surface. As the overall size of the cover 5 with the microfluidic channel 6 is around 500 μm to 1 mm, disposing it on the chip 2 requires the use of a chip with a large area, and loses the advantages of miniaturization of the chip 2. Hence, another microfluidic channel detection system 10 is developed, as described below.

As shown in FIG. 2, this shows another conventional microfluidic channel detection system 10. This conventional microfluidic channel detection system 10 has an inactive layer 60 covering a chip 20 and wires 40, and an opening exposing a sensing region of the chip 20. A cover 50 having a microfluidic channel 52 is disposed on top of the inactive layer 60. Since the cover 60 having the microfluidic channel 52 is not disposed on top of the chip 20, and does not occupy the surface area of the chip 20, the chip 20 still maintains the advantages of miniaturization. However, the flow path of the fluid specimen through the chip 20 and the adjacent region in the microfluidic channel 52 in this system is not smooth. As shown in FIG. 2, the arrows in the figure show the flowing directions of the fluid specimen. When the fluid specimen flows through the sensing region of the chip during measurements, the fluid specimen does not completely flow in the same direction, causing a phenomenon of flow field disturbance, and resulting in unstable measurements.

### SUMMARY OF THE INVENTION

The objective of the present invention is to provide a microfluidic channel detection system, in which a plane formed by a first surface where a sensing region of a chip is located and an inactive layer is used to resolve a problem of unevenness between the chip and the adjacent region in a conventional microfluidic channel detection system.

To achieve the above objective and overcome the shortcomings of prior arts, the present invention provides a microfluidic channel detection system, including a chip 110 for sensing, a substrate, a first inactive layer, an electrical connection member, and a cover having a microfluidic channel.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a schematic side view illustrating the structure of a conventional microfluidic channel detection system;

FIG. 2 is a schematic side view illustrating another conventional microfluidic channel detection system and the condition of the fluid flowing in the microfluidic channel thereof.

FIG. 3A is a schematic top view of the microfluidic channel detection system in accordance with the first embodiment of this invention; FIG. 3B is a schematic side cross-sectional view of the microfluidic channel detection system taken along line AA′ in FIG. 3A; FIG. 3C is a schematic side cross-sectional view of the microfluidic channel detection system taken along line BB′ in FIG. 3A.

FIG. 4A is a schematic top view of the microfluidic channel detection system in accordance with the second embodiment of this invention; FIG. 4B is a schematic side cross-sectional view of the microfluidic channel detection system taken along line AA′ in FIG. 4A;

FIGS. 5A-5I are schematic side views of assembly steps of the microfluidic channel detection system in accordance with the first embodiment of the invention.

FIGS. 6A-6C are schematic side views of manufacturing steps of the cover of the microfluidic channel detection system in accordance with the invention.

FIGS. 7A-7F are schematic side views of assembly steps of the microfluidic channel detection system in accordance with the second embodiment of the invention.

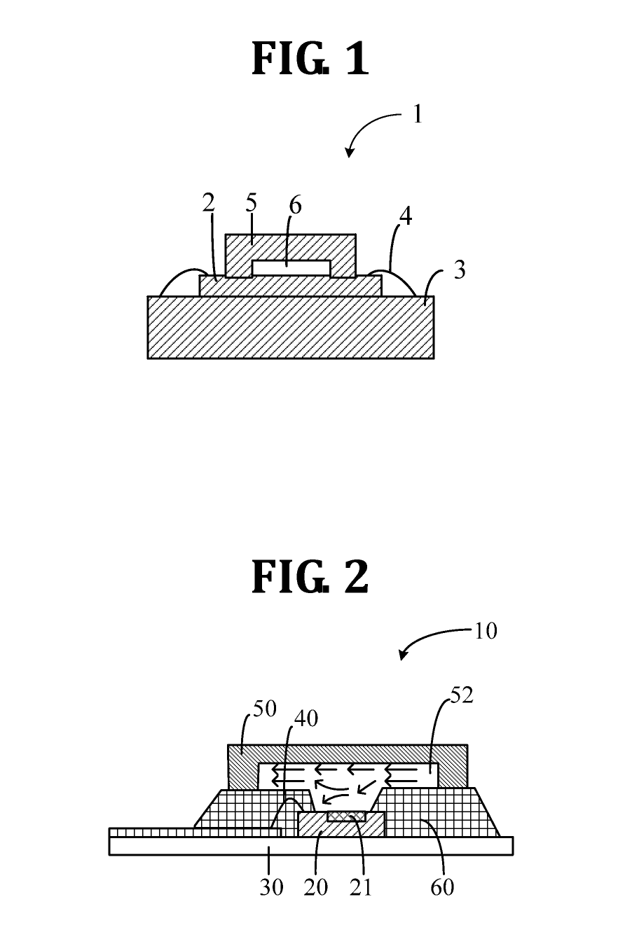

### DETAILED DESCRIPTION OF THE PREFERRED EMBODIMENTS

By reference to the accompanying drawings, the technological content and embodiments of the present invention are described in detail as follows:

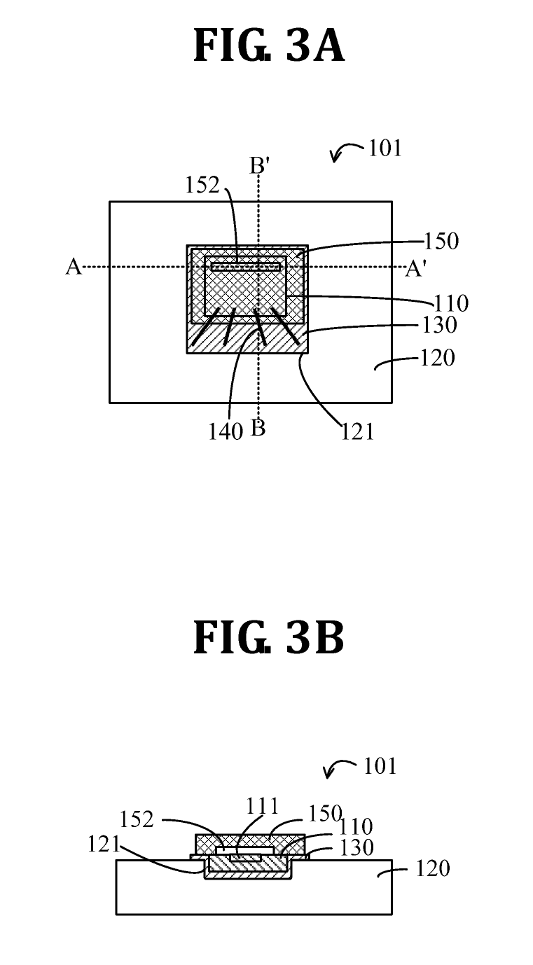

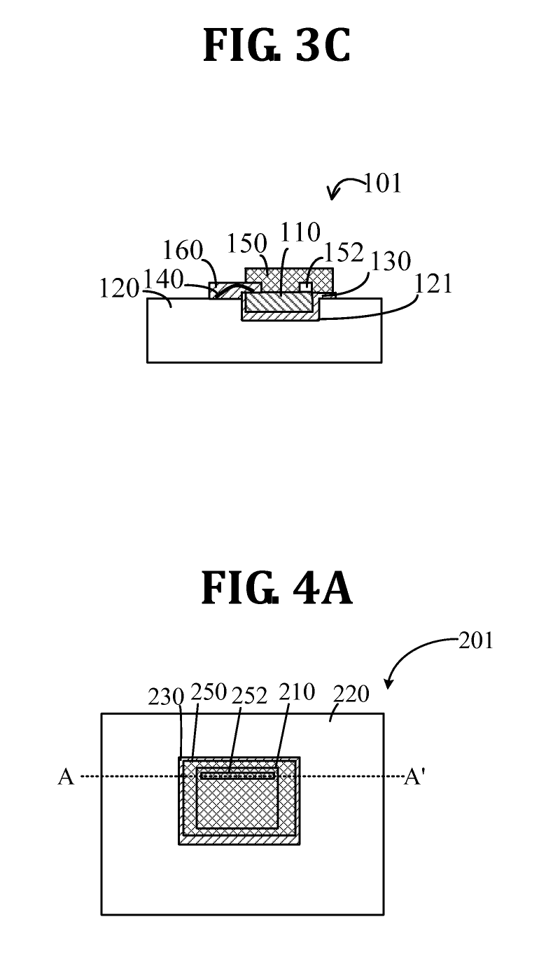

Referring to FIGS. 3A-3C, these are schematic top view and side cross-sectional views from different directions of the present microfluidic channel detection system 101 in accordance with the first embodiment of this invention. The microfluidic channel detection system 101 of the present invention includes a chip 110 for sensing, a substrate 120, a first inactive layer 130, an electrical connection member 140, and a cover 150 having a microfluidic channel 152. The chip 110 has a first surface, a second surface opposite to the first surface, and a sensing area 111 located on the first surface of the chip 110 for sensing an analyte in a test specimen. The sensing region 111 is a region of the chip 110 in contact with the analyte in the test specimen. The substrate 120 bears the whole micro-channel detection system 101, and has a recess 121 sized to accommodate the chip 110. In practice, for accommodating the chip 110, the width of the recess 121 is usually wider than the chip 110, the depth being deeper than the thickness of the chip 110. The chip 110 is disposed into the recess 121, so that the first surface and the sensing region 111 are exposed from the opening of the recess 121, and the second surface faces the bottom of the recess 121. The first inactive filler layer 130 fills the gaps between the substrate 120 and the chip 110 within the recess 121 of the substrate 120, and between the second surface of the chip 110 and the bottom of the recess 121, and surrounds the periphery of the chip 110 on the substrate 120, so that the first inactive layer 130 and the first surface of the chip 110 constitute a plane, and the sensing area 111 is located in the plane and exposed. The cover 150 having the microfluidic channel 152 is disposed on top of the plane constituted with the first inactive layer 130 and the first surface of the chip 110, so that the test specimen flows through the microfluidic channel 152 and contacts the sensing region 111, and the chip 110 senses the analyte in the test specimen to send a signal. The electrical connection member 140 is electrically connected to the chip 110 for transferring the signal from the chip 110 to the outside. Since the plane constituted with the first inactive layer 130 and the first surface is flat, the test specimen is allowed to flow smoothly through the sensing region 111. The detection results are not interfered with by the flow field disturbance resulting from unevenness between the chip surface and the adjacent region, so as to improve the accuracy of the detection results.

The first inactive layer 130 is any material with a property of plasticity, thermosetting or thermoplasticity, e.g., a polymer material, an organic material, or an inorganic material. The plasticity described above refers to a property in which a solid material, when force is applied, undergoes deformation and remains. The aforementioned thermosetting refers to that a solid material with plasticity property is irreversibly converted into a solid lacking plasticity after being solidified/cured by the action of heat or suitable radiation. Thermoplasticity described above means a material with plasticity turns to a solid state lacking plasticity upon cooling, or a material lacking plasticity becomes plastic, pliable or moldable upon heating. In the polymer material, polydimethylsiloxane (PDMS) has properties of decent plasticity, thermosetting, transparency, biocompatibility, and a relatively low cost. In addition, the bonding technique used to bond two PDMS materials together has become quite mature, so PDMS is preferably used as the material of the first inactive layer 130 in this embodiment. However, this is an exemplary embodiment and should not be used to limit the scopes of the claims.

The electrical connection member is implemented as one or multiple first wire(s) 140 and disposed on top of the plane constituted by the first surface of the chip 110 and the first inactive layer 130, so as to electrically connect circuits on the substrate to the chip 110. If the first wire 140 were directly exposed to the external environment, it could be interfered with, damaged or hydrolyzed in a solution. In order to solve this problem, the microfluidic channel detection system of the present invention further comprises a second inactive layer 160 covering and protecting the first wire 140. The second inactive layer 160 is any material with a plasticity, thermosetting or thermoplasticity, e.g. a polymer material, an organic material, an inorganic material, which can be the same material as the first inactive layer 130 or a different material from the first inactive layer 130. For the same reasons as previously described, polydimethylsiloxane (PDMS) is preferably used as the material of the second layer 160, the same as the material of the first layer 130 in this embodiment.

In the present embodiment, the substrate 120 is a printed circuit board (PCB) in the present embodiment, and a material selected from a group consisting of silicon, semi-fiber, fiber, glass fiber, glass wool, aluminum nitride, aluminum oxynitride, ceramic, PTFE (polytetrafluoroethene), flexible materials, glass, polymers and plastics. The cover 150 having the microfluidic channel 152 is a material selected from a group consisting of the photoresist, glass, polymers, and plastics. In the present embodiment, polydimethylsiloxane (PDMS) is preferably used as the material for the cover 150 having the microfluidic channel 152. The chip 110 is a material selected from a group consisting of silicon (Si), germanium (Ge), silicon carbide (SiC), aluminum arsenide (AlAs), aluminum phosphide (AlP), aluminum antimonide (AlSb), nitride boron (BN), boron phosphide (BP), gallium arsenide (GaAs), gallium nitride (GaN), gallium antimonide (GaSb), indium arsenide (InAs), indium phosphide (InP), indium antimonide (InSb), cadmium sulfide (CdS), cadmium selenide (CdSe), cadmium telluride (CdTe), zinc oxide (ZnO), zinc sulfide (ZnS), zinc selenide (ZnSe), selenium tellurium (ZnTe), mercuric sulfide (HgS), mercury selenide (HgSe), mercury telluride (HgTe), lead sulfide (PbS), lead telluride (PbTe), glass, polymers, and plastics. In this embodiment, silicon is used as the material of the chip 110, and a complementary metal oxide semiconductor integrated circuit chip (CMOS IC Chip) is preferably used on account of the characteristics of low power consumption and less heat production. The material of each element described above and the chip types used are only the embodiments of the present invention, and should not be used to limit the scopes of the claims.

In addition, the microfluidic channel detection system further includes a valve and a pump or a mixer disposed on the substrate 120 and connected to the microfluidic channel 152 to provide the convenience and functionality for the system in this embodiment of the present invention (not shown).

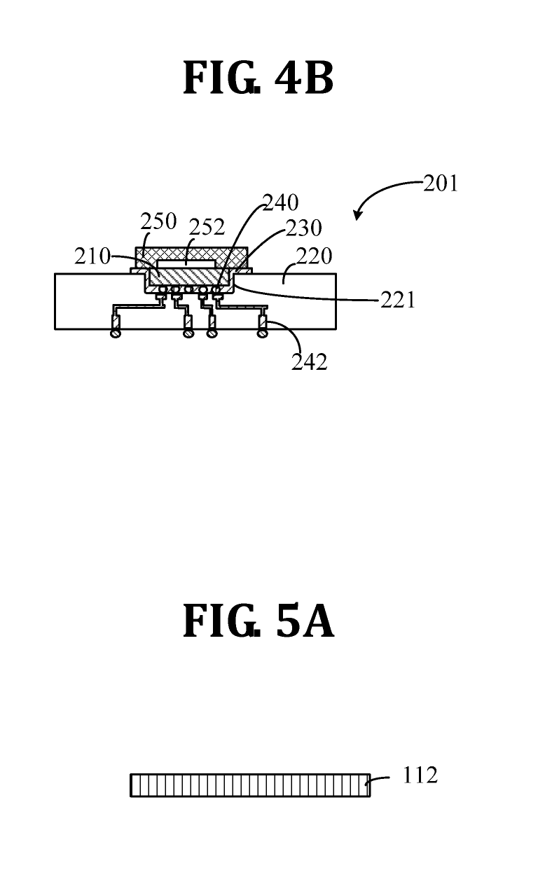

Referring to FIGS. 4A and 4B, these are a schematic top view and a side cross-sectional view of the microfluidic channel detection system 201 in accordance with the second embodiment of this invention. The elements of this embodiment are similar to those of the first embodiment, including a chip 210, a substrate 220, a first inactive layer 230, an electrical connection member 240, and a cover 252 having a microfluidic channel 250. The difference between the microfluidic channel detection system 201 in the present embodiment and the previous embodiment is that the electrical connection member 240 is a conductive ball grid array 240 disposed between the second surface of the chip 210 and the recess 221 bottom of the substrate 220, and one or more wire(s) are embedded in the substrate for connecting the conductive ball grid array 240. Tin (Sn) is preferably used as the material of the conductive ball grid array 240 while silicon (Si) is preferably used as the material of the substrate 220. Nevertheless, this is only an exemplary implementation, and should not be used to limit the scopes of the claims. The other elements, including the chip 210, the substrate 220, the first inactive layer 230 and the cover 250 having the microfluidic channel 252 are the same as those in the first embodiment.

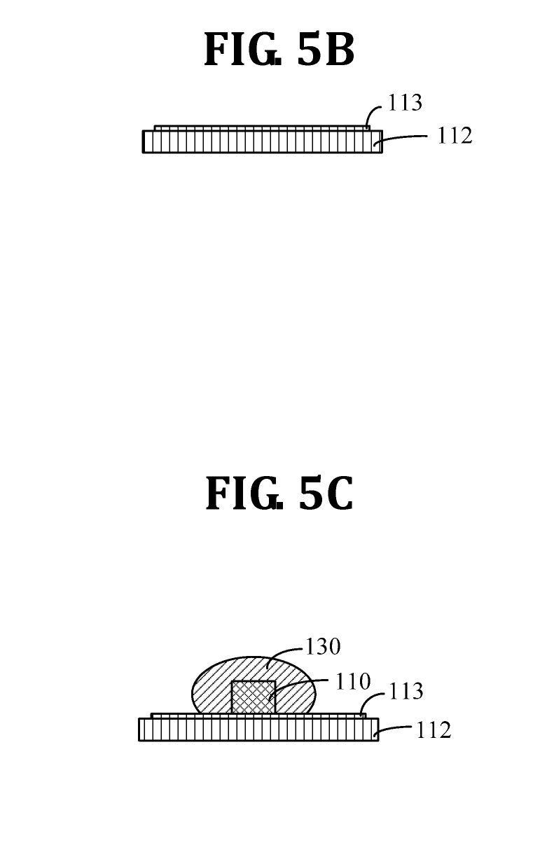

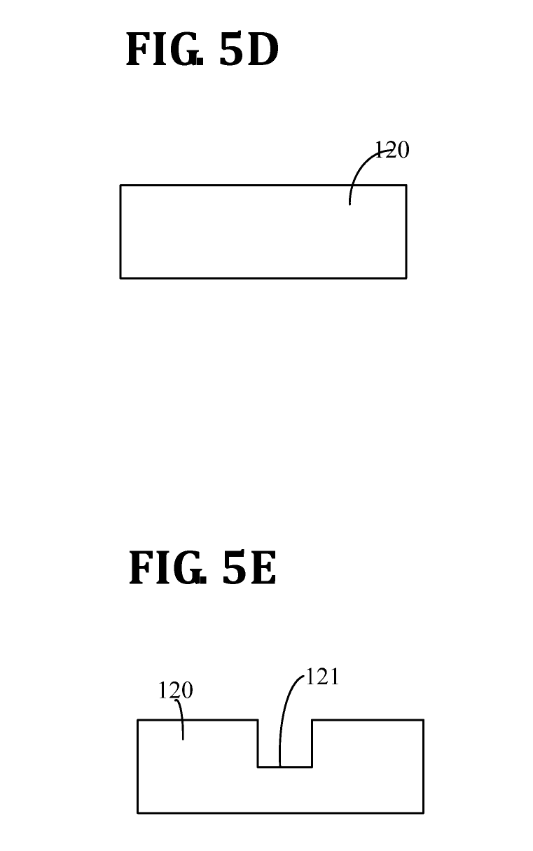

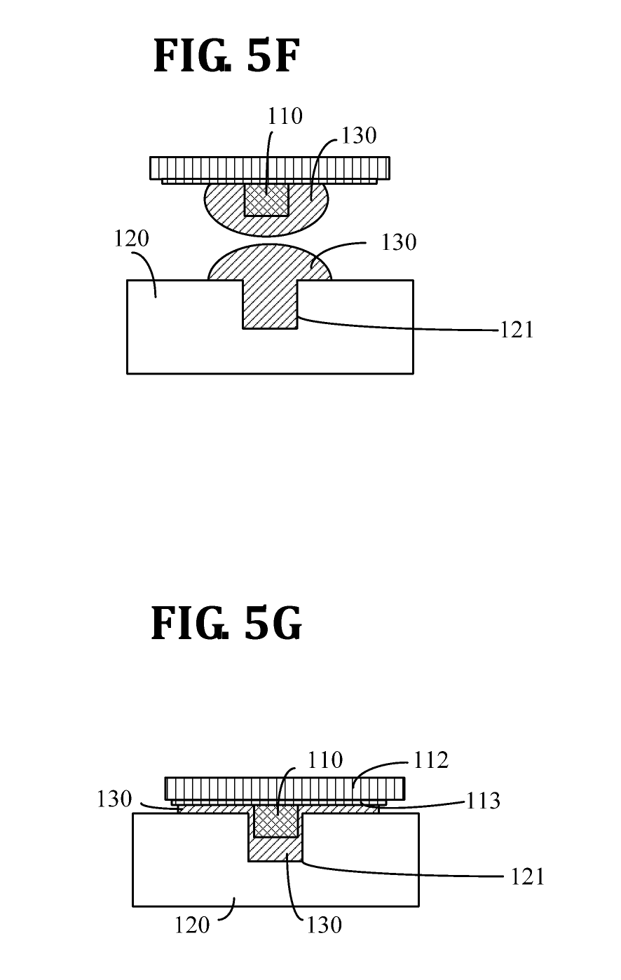

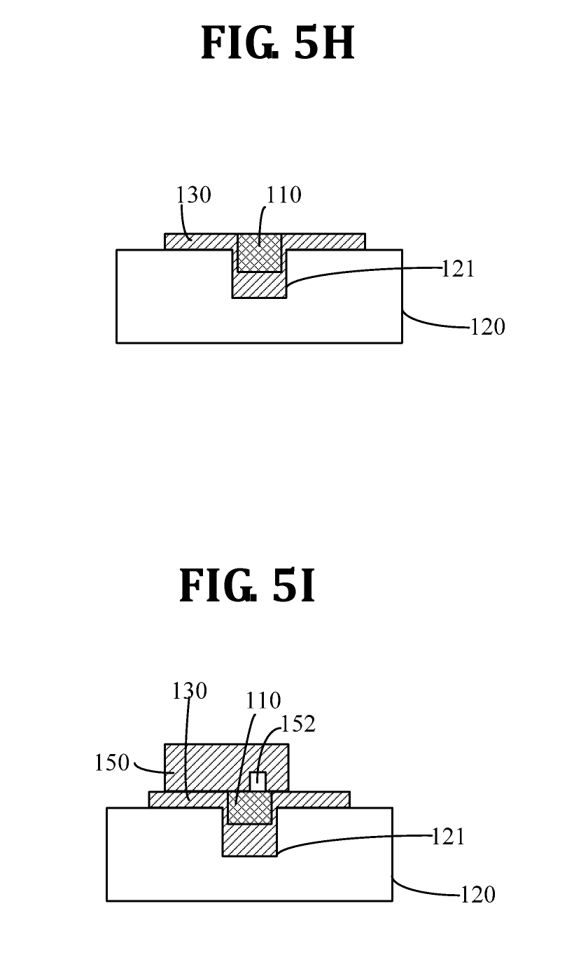

Referring to FIGS. 5A-5I, these are schematic side views of the manufacturing process of the microfluidic channel detection system 101 according to the first embodiment of the present invention. The manufacturing process of the microfluidic channel detection system primarily includes the following steps: As shown in FIG. 5A, a plate 112 is prepared, whose material is acrylic glass (poly methyl methacrylate, PMMA), but is not limited thereto. As shown in FIG. 5B, an insulating layer 113 is coated on the plate 112, which is a silicone rubber layer, but should not be used to limit the scope of the claim. As shown in FIG. 5C, a chip 110 for sensing is adhered to the insulating layer 113 of the plate 112. The chip 110 has a first surface, a second surface opposite to the first surface, and a sensing region 111 located on the first surface. The first surface of the plate 113 contacts the insulating layer 113, and a first inactive layer 130 in a soft solid or viscous state covers five surfaces, except for the first surface of the chip 110. As shown in FIGS. 5D-5E, a substrate 120 is prepared, and a computer numerical control engraving machine (Computer Numerical Control Carving Machine, CNC Carving Machine) is used to carve out a recess 121 wider and deeper than the chip 110 on one side of the substrate 120. As shown in FIG. 5F, the same inactive layer 130 in a soft-solid or viscous state covers the recess 121. As shown in FIGS. 5F and 5G, the chip 110 is put within the recess 121 by facing the surface of the plate 112 where the chip 110 is adhered to the side of the substrate 120 where the recess 121 is formed, aligning the chip 110 with the recess 121, and placing the plate 112 on the substrate 120. During the process of placing the chip 110 into the recess 121, the chip 110 is prevented from contacting the recess 121 wall to cause deviation. The first inactive layer 130 in a solid or vicious state exists in the gaps between the recess 122 wall and the chip 110 and between the plate 112 and the substrate 120 at this time. As shown in FIG. 5G, the first inactive layer 130 is solidified/cured through placing the combination of the substrate 120 and the plate 112 on a heating platform, and heating to 70° C. for 30 minutes. As shown in FIG. 5H, after the solidification/curing is complete, the plate 112 is removed, and the insulating layer 113 facilitates the chip 110 being detached from the plate 112 and staying within the recess 121. The second surface of the chip 110 faces the recess 121, the first surface is exposed from the opening of the recess 121, and the first surface where the sensing region 111 of the chip 110 is located and the first inactive layer 130 constitutes a plane together. Moreover, the plane is close to the surface of the substrate 120 where the recess 121 is formed, so that the distance between the two is usually less than 0.5 mm for reducing the flow field disturbance. In this embodiment, one or more first wire(s) 140 are further disposed on the plane and the surface of the substrate 120 as a electrical connection member 140 for connecting the chip 110, and transmitting a signal detected by the chip 110 to the outside. Subsequently, a second inactive layer 160 is used for covering and protecting the first wire 140 from damage and interference from the external environment (not shown). As shown in FIG. 5I, a cover 150 with a microfluidic channel 152 prepared and aligned with the sensing region 111 of the chip 110 under a microscope is fixed on top of the plane constituted by the first surface of the chip 110 and the first inactive layer 130. Prior to fixing the cover 150 having the microfluidic channel 152 on top of the plane, the manufacturing method further comprises executing surface modification on bonding positions of the cover and the plane with an oxygen plasma for enhancing the bonding strength between the cover 150 having the microfluidic channel 152 and the plane (not shown). After the entire microfluidic channel detection system is complete, it can be further provided with a valve, a pump, or a mixer connected to the microfluidic channel for increasing the convenience and functionality for this system (not shown in the figures).

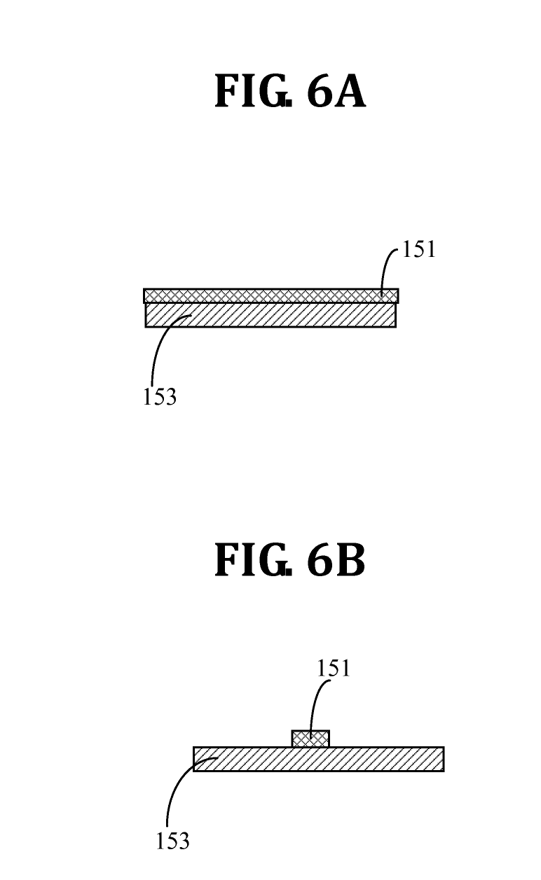

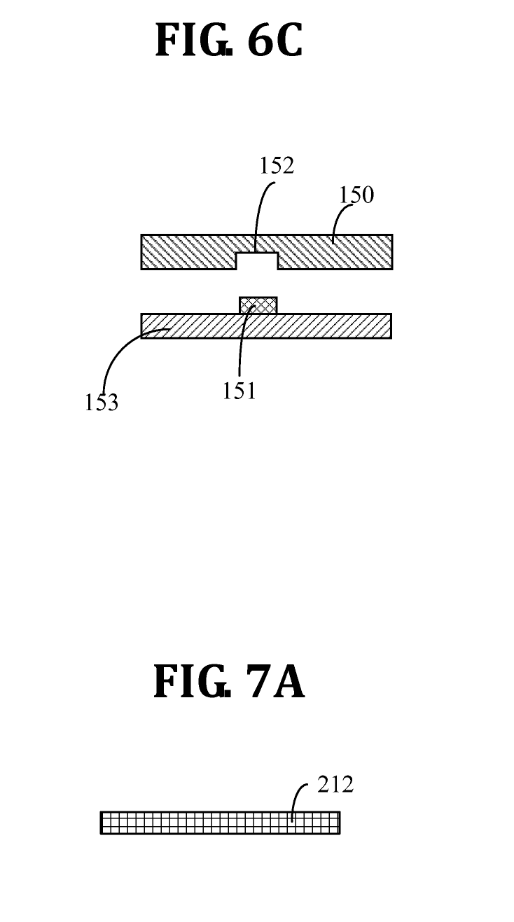

Referring to FIGS. 6A-6C, these are schematic side views of manufacturing steps of the cover 150 having the microfluidic channel 152 in the microfluidic channel detection system 101 according to the present invention. In this first embodiment, the microfluidic channel 152 of the cover 150 is shaped through imprinting a mold having the microfluidic channel pattern formed by photoresist 151 on the cover 150 with the microfluidic channel 152. The specific manufacturing steps of the cover 150 with the microfluidic channel 152 are as follows: As shown in FIG. 6A, a negative photoresist 151 is coated on the mold base plate 153. In this embodiment, the negative photoresist is SU-8 photoresist 151, and the mold base plate 153 is made of glass, but these are exemplary implementations, and should not be used to limit the scopes of the claims. As shown in FIG. 6B, the negative photoresist 151 is masked, only the region which will form the microfluidic channel pattern is revealed and then exposed to light. The masked region without exposure is solved in a developer, and the revealed region is insoluble in the developer because of crosslinking and solidification resulted from exposure. Therefore, the mold base plate 153 and the exposed photoresist 151 together form the mold with a microfluidic channel pattern. As shown in FIG. 6C, this mold is imprinted on the cover 150 with the microfluidic channel 152 in a soft solid or viscous state, the solidification/curing is conducted, and then the mold is removed to shape the cover 150 with the microfluidic channel 152.

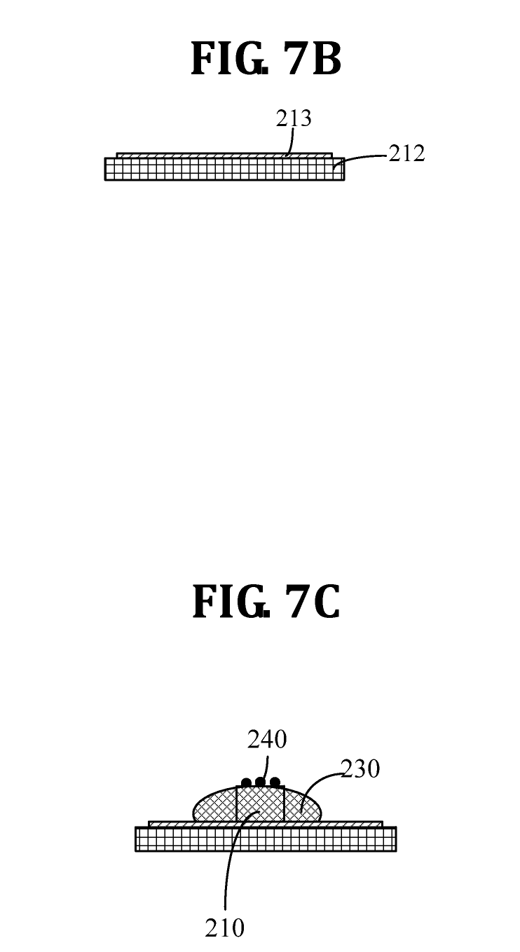

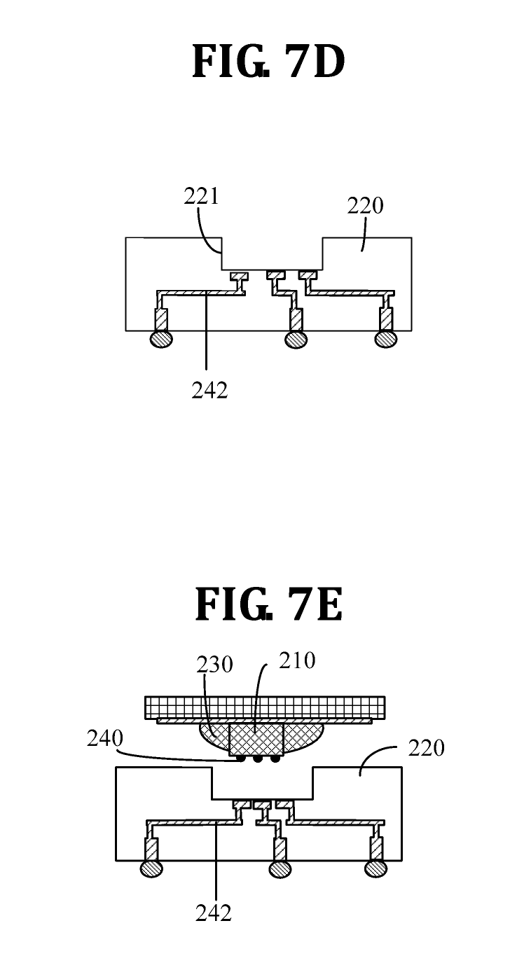

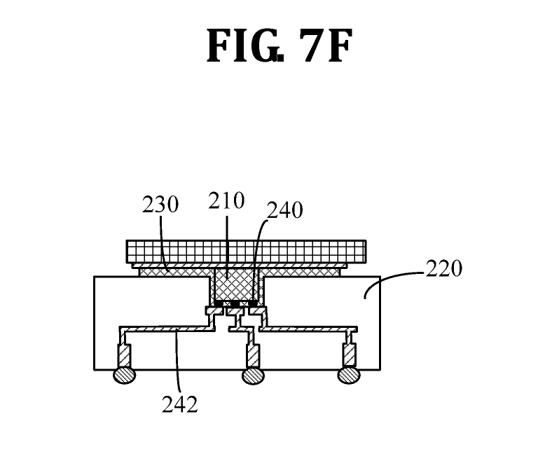

Referring to FIGS. 7A to 7F, these are schematic side views of the microfluidic channel detection system 201 in accordance with the second embodiment of the present invention. The assembly steps of the microfluidic channel detection system in the second embodiment of the present invention 201 are similar to those in the first embodiment, except that the electrical connection member is implemented as a conductive ball grid array 240 and disposed on the second surface of the chip 210. In FIGS. 7A and 7B, a plate 212 is prepared and coated with an insulating layer 213, the same as in the first embodiment. As shown in FIG. 7C, a first inactive layer 230 in a soft solid or viscous state covers the chip 210, but only the four surfaces other than the first surface and the second surface, not the conductive ball grid array 240 on the second surface. As shown in FIG. 7D, the substrate 220 is prepared and embedded with second wires 242, whose terminals are exposed from the bottom of the recess 221. Consequently, the first inactive layer 230 in a soft solid or viscous is not used to cover the recess 221 for preventing it from blocking the electrical connection between the conductive ball grid array 240 and the terminals of the second wires 242 embedded in a substrate 220. As shown in FIG. 7E, when the chip 210 is put into the recess 221, the conductive ball grid array on the chip 210 is electrically connected to the terminals of the second wires 240 embedded in the second substrate, and transmits a signal sensed by the chip 210, replacing the first wire 140 disposed on top of the plane and the substrate 120 surface in the first embodiment. The remaining steps are the same as in the first embodiment.

In summary, the technical features of the present invention are utilizing a plane constituted by an inactive layer and a surface of a chip to cause a specimen in a microfluidic channel to flow smoothly, to correct flow field disturbances resulted from unevenness between the chip and the adjacent region in a conventional microfluidic channel detection system, and to enhance the accuracy of the microfluidic channel detection system of the present invention.

The present invention has been described with a preferred embodiment thereof and it is understood that various modifications, without departing from the spirit of the present invention, are in accordance with the embodiments of the present invention. Hence, the embodiments described are intended to cover the modifications within the scope and the spirit of the present invention, rather than to limit the present invention.

What is claimed is:

1. A microfluidic channel detection system, comprising:
a chip having a first surface where a sensing region is located and a second surface opposite to the first surface;
a substrate having a recess for containing the chip, so that the second surface of the chip faces the recess and the first surface is exposed;
a first inactive layer filling gaps between the chip and the substrate in the recess of the substrate, and surrounding the circumference of the chip on the substrate, so as to form a plane with the first surface of the chip;
an electrical connection member electrically connected to the chip; and
a cover having a microfluidic channel and being disposed on top of the plane formed by the chip and the first inactive layer.

2. The microfluidic channel detection system as claimed in claim 1, wherein said electrical connection member is a wire disposed on top of the plane formed by the chip and the first inactive layer and electrically connected to the chip.

3. The microfluidic detection system as claimed in claim 2, further comprising a second inactive layer covering the wire.

4. The microfluidic detection system as claimed in claim 3, wherein the second inactive layer comprises a material identical to a material of the first inactive layer.

5. The microfluidic detection system as claimed in claim 1, wherein said chip comprises a material selected from a group consisting of silicon (Si), germanium (Ge), silicon carbide (SiC), aluminum arsenide (AlAs), aluminum phosphide (AlP), aluminum antimonide (AlSb), boron nitride (BN), boron phosphide (BP), gallium arsenide (GaAs), gallium nitride (GaN), gallium antimonide (GaSb), indium arsenide (InAs), indium phosphide (InP), indium antimonide (InSb), cadmium sulfide (CdS), cadmium selenide (CdSe), cadmium telluride (CdTe), zinc oxide (ZnO), zinc sulfide (ZnS), zinc selenide (ZnSe), selenide, telluride (ZnTe), mercuric sulfide (HgS), mercury selenide (HgSe), mercury telluride (HgTe), lead sulfide (PbS), lead telluride (PbTe), glass, polymers, and plastics.

6. The microfluidic detection system as claimed in claim 1, wherein said first inactive layer is further disposed between the second surface of the chip and the recess of the substrate.

7. The microfluidic detection system as claimed in claim 1, wherein said chip is a complementary metal-oxide-semiconductor integrated circuit chip (CMOS IC Chip).

8. The microfluidic detection system as claimed in claim 1, wherein said substrate comprises a material selected from a group consisting of silicon, semi-fiber, fiber, glass fiber, glass wool, aluminum nitride, aluminum oxynitride, ceramic, PTFE (polytetrafluoroethene), flexible materials, glass, polymers, and plastics.

9. The microfluidic detection system as claimed in claim 1, wherein said cover having the mircrofluidic channel comprises a material selected from a group consisting of photoresist, glass, polymers, and plastic materials.

10. The microfluidic detection system as claimed in claim 9, wherein said polymer is polydimethylsiloxane (PDMS).

11. The microfluidic detection system as claimed in claim 1, wherein said first inactive layer comprises a material selected from a group consisting of polymers, organic materials, and inorganic materials.

12. The microfluidic detection system as claimed in claim 11, wherein said polymer is polydimethylsiloxane (PDMS).

13. The microfluidic detection system as claimed in claim 1, further comprising a valve, a pump or a mixer disposed on the substrate and connected to the microfluidic channel.

14. The microfluidic detection system as claimed in claim 1, wherein said electrical connection member is a conductive ball grid array disposed between the second surface of said chip and the recess of the substrate, and a wire is embedded in the substrate for connecting the conductive ball grid array.

15. A method of manufacturing a microfluidic channel detection system, comprising:
providing a plate having a chip attached on a surface thereof, the chip having a first surface where a sensing region is located and a second surface opposite to the first surface, and the first surface of the chip contacts said plate;
providing a substrate having a recess formed in one side thereof;
covering at least one of the chip and the recess with a first inactive layer;
placing the plate on the substrate by facing the surface of the plate where the chip is attached to the side of the substrate where the recess is formed, so as to put the chip into the recess;
solidifying the first inactive layer, removing the plate and leaving the chip within the recess, so that the second surface of the chip faces the recess, the first surface of is exposed, and the first surface of the chip where the sensing area is located and the first inactive layer form a plane together; and
disposing a cover having a microfluidic channel on top of the plane formed by the chip and the first inactive layer to align the microfluidic channel with the sensing region.

16. The manufacturing method as claimed in claim 15, wherein the surface of said plate is coated with an insulating layer, and the chip is attached to the insulating layer.

17. The manufacturing method as claimed in claim 16, wherein the insulating layer is a silicon rubber layer.

18. The manufacturing method as claimed in claim 15, further comprising disposing a wire on top of the plane and the substrate and covering the wire with a second inactive layer before disposing the cover on top of the plane.

19. The manufacturing method as claimed in claim 15, wherein the microfluidic channel of said cover is formed by imprinting a mold having a pattern of the microfluidic channel formed by a photoresist onto said cover.

20. The manufacturing method as claimed in claim 15, further comprising executing surface modification on bonding positions of the cover and the plane with an oxygen plasma prior to disposing the cover on top of the plane.

21. The manufacturing method as claimed in claim 15, wherein said chip is formed by a material selected from a group consisting of silicon (Si), germanium (Ge), silicon carbide (SiC), aluminum arsenide (AlAs), aluminum phosphide (AlP), aluminum antimonide (AlSb), boron nitride (BN), boron phosphide (BP), gallium arsenide (GaAs), gallium nitride (GaN), gallium antimonide (GaSb), indium arsenide (InAs), indium phosphide (InP), indium antimonide (InSb), cadmium sulfide (CdS), cadmium selenide (CdSe), cadmium telluride (CdTe), zinc oxide (ZnO), zinc sulfide (ZnS), zinc selenide (ZnSe), selenide, telluride (ZnTe), mercuric sulfide (HgS), mercury selenide (HgSe), mercury telluride (HgTe), lead sulfide (PbS), lead telluride (PbTe), glass, polymers, and plastics.

22. The manufacturing method as claimed in claim 15, wherein said chip is implemented as a complementary metal-oxide-semiconductor integrated circuit chip (CMOS IC Chip).

23. The manufacturing method as claimed in claim 15, wherein said substrate is formed by a material selected from a group consisting of silicon, semi-fiber, fiber, glass fiber, glass wool, aluminum nitride, aluminum oxynitride, ceramic, PTFE (polytetrafluoroethene), flexible materials, glass, polymers and plastics.

24. The manufacturing method as claimed in claim 15, wherein said cover having mircrofluidic channel is formed by a material selected from a group consisting of photoresist, glass, polymers, and plastic material.

25. The manufacturing method as claimed in claim 24, wherein the polymer is polydimethylsiloxane (PDMS).

26. The manufacturing method as claimed in claim 15, wherein said first inactive layer is formed by a material selected from a group consisting of polymers, organic materials, and inorganic materials.

27. The manufacturing method as claimed in claim 26, wherein said polymer is polydimethylsiloxane (PDMS).

28. The manufacturing method as claimed in claim 15, further comprising disposing a valve, a pump, or a mixer connected to the microfluidic channel.

29. The manufacturing method as claimed in claim 15, wherein a conductive ball grid array is disposed on the second surface of the chip, and a wire is embedded in the substrate for connecting the conductive ball grid array.

30. The manufacturing method as claimed in claim 29, wherein said first inactive layer covers said chip but exposes the second surface where the conductive ball grid array is located before the chip is put into the recess.
